(19) United States
(12) Patent Application Publication
(10) Pub. No.: US 2014/0316319 A1
(43) Pub. Date: Oct. 23, 2014

(54) PELVIC COMPRESSION POSITIONING DEVICE AND METHOD OF USE

(71) Applicant: Edward BANNISTER, Claremont, CA (US)

(72) Inventor: Edward BANNISTER, Claremont, CA (US)

(21) Appl. No.: 14/260,133

(22) Filed: Apr. 23, 2014

Related U.S. Application Data
(60) Provisional application No. 61/815,137, filed on Apr. 23, 2013.

(51) Int. Cl. A61H 1/00; A61F 5/01

(52) U.S. Cl. CPC A61H 1/006; A61F 5/0102
USPC 602/19

(57) ABSTRACT

A pelvic compression positioning device and method are provided. The pelvic compression device comprises an extension handle, a friction shield attached to the extension handle, the friction shield being releasably connected to a bracing portion of an orthotic device, wherein, the extension handle and the friction shield are slid underneath a fractured body part of a patient without substantially moving the fractured body part.

### RELATED APPLICATIONS

This application claims the benefit of priority to U.S. Provisional Application No. 61/815,137 entitled PELVIC COMPRESSION POSITIONING DEVICE AND METHOD OF USE, FILED ON Apr. 23, 2013, and is related to U.S. patent application Ser. No. 09/420,408 titled ORTHOTIC TRAUMA DEVICE filed on Oct. 19, 1999, now issued as U.S. Pat. No. 6,602,214. The entirety of the prior applications is expressly incorporated by reference herein.

### FIELD OF THE INVENTION

Aspects of the present invention relate to pelvic compression positioning devices and methods of use thereof. Specifically, aspects of the present invention relate to pelvic compression positioning devices for use in conjunction with orthotic devices for the emergency treatment of bone fractures. More particularly, the pelvic compression positioning devices in accordance with aspects of the present invention may be used to facilitate positioning of orthotic devices for partial or substantial immobilization of bone fractures, including pelvic fractures, in emergency rooms of hospitals, at an accident site or in transit from an accident site, where quick and effective stabilization of a fracture is essential and where such a device must be adapted both to the dimensions of the individual and the body part being treated.

### BACKGROUND

A common method of treating fractures in the field or at an accident scene is to determine the nature and general extent of the injury and thereafter attempt to immobilize the fractured body part to avoid further injury to the patient, should the body part be moved. Although it may be appropriate to immobilize bone fractures of a limb with a cast arrangement at an accident scene, because of the nature of the type of materials used to form such casts and the time required to apply and subsequently remove the cast when the patient reaches the hospital or other treatment facility, it is generally not feasible to attempt to immobilize pelvic fractures by such means. In addition, without the benefit of X-ray pictures of the injured area, the fracture could be improperly immobilized, causing additional harm. Little more can be done in the field since the means to produce X-ray pictures of the fracture at the accident scene are usually unavailable. Should the patient be bleeding, additional steps may be taken to arrest bleeding.

Although exceptions exist, the time elapsed between the occurrence of the fracture of a limb and the subsequent treatment of the limb in a modern medical facility does not result in a life threatening situation as long as the treatment in such a facility is not unduly delayed, the patient has not lost a large volume of blood, and initial emergency treatment has been administered in the interim. However this is not true of bone fractures in other parts of the body, particularly in the pelvic region.

Tens of thousands of pelvic fractures occur each year. Most of these injuries result from motor vehicle collisions, falls from height, or industrial accidents. Many of these injuries are life threatening with severe blood loss from torn blood vessels or bleeding from fractured pelvic bones. Even with hairline pelvic fractures, blood loss may be significant since the pelvis bones are very porous.

Means of stabilization of pelvic fractures by emergency medical service technicians for transport of the patients from the accident scene are described in more detail in U.S. Pat. No. 6,602,214, which is incorporated by reference herein. Such means include a device and a method of rapidly immobilizing a fractured bone and particularly a fractured pelvic bone, and ensure in most instances that a supply of sufficient pressure at the site of the fracture is applied to diminish or arrest bleeding that may accompany the fracture. In addition, such means may be left in place both during the early stages of emergency treatment and through initial assessment of the degree of injury and proposed treatment, including conducting an X-ray examination, CT scan, MRI scan, among others.

Compression of most types of bone fractures, including a fractured pelvis, with the orthotic device described in U.S. Pat. No. 6,602,214 stabilizes the broken bones of the pelvis and helps control blood loss. Reducing the motion of the bone fragments allows bleeding sites to clot off helping to limit blood loss as well as greatly improving the comfort of the patient. Use of this device lessens the likelihood of further injury to nerves, blood vessels or internal organs when the patient is moved. Further, this device provides sufficient provisional stabilization of the pelvis such that that the need for emergent application of a pelvic external fixator is eliminated in most cases. Such orthotic devices include an orthosis body, interchangeably referred to herein as a bracing portion, in the shape of a wide belt, adapted to be wrapped around a portion of a body of a patient, the bracing portion being elongated and formed from a material which is easily cut with cloth-cutting scissors. A detachable fastening device is provided at the distal or outer ends of the elongated bracing portion to detachably secure the ends around the part of the body where the bone fracture is located. The fastening device may be provided with a means for adjusting the tightness or tension of the bracing portion, accomplished by increasing the mechanical advantage of the fastening device, for example. The means for increasing the mechanical advantage of the fastening device may be a detachable pulley system, such as the one described in U.S. Pat. No. 6,602,214, including a pair of pulley banks operatively connected via a cable, such that the two pulley banks are in a juxtaposed relationship, the first of the pulley banks in the pair being detachably disposed on a first distal end of the elongated bracing portion and the second pair being detachably disposed on a second distal end of the elongated bracing portion. However, other means, such as hook and loop fabric, known as Velcro®, or other similar means for increasing the mechanical advantage may also be used to achieve similar tightening of the orthotic device as the detachable pulley system described in U.S. Pat. No. 6,602,214.

However, in order to secure the bracing portion around the body of a patient with a hip or pelvic fracture, for example, it is typically necessary to lift the patient and to slide or otherwise insert the bracing portion under the small back of the patient, thereby not only causing additional discomfort to the patient, but also risking misaligning portions of the fractured bones prior to securing the bracing portion around the pelvic area of the patient. Such misalignment may have severe consequences, such as additional bleeding, preventing proper alignment of the fractured bone fragments after the patient is taken to the hospital, or severing the spinal cord, for example.

In order to avoid causing further patient discomfort and risking misalignment of the fractured bone fragments, there is a need in the art for a device and method of using such a device that facilitates positioning the bracing portion under the patient's fractured body part, such as a pelvis, without the need to lift the body part in order to slide or otherwise insert the bracing portion underneath the patient.

In addition, it is an object of the present invention to require a skill level of the emergency personnel applying such a device to a patient suffering from a bone fracture which is not significantly higher than is generally required from such personnel at the present time in treating the same type of fracture using currently available means for such treatment.

It is yet another object of the present invention to provide a pelvic compression positioning device for easy insertion of an orthotic device under a patient's fractured body part, such as a pelvis, without the need to lift the fractured body part, while being able to remove the device once the bracing portion extends on both sides of the fractured body part to enable fastening and securing of the bracing portion around the patient's fractured body part via a fastening device.

### SUMMARY

The following presents a simplified summary of one or more aspects of the present invention in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

Aspects of the present invention solve the above-identified needs, as well as others, by providing a device and method of using such a device that facilitates positioning the bracing portion of an orthotic device under the patient's fractured body part, such as a pelvis, without the need to lift the body part in order to slide or otherwise insert the bracing portion underneath the patient.

In addition, aspects of the present invention provide a device and method of using such a device that do not require a skill level of the emergency personnel applying such a device to a patient suffering from a bone fracture to be significantly higher than is generally required from such personnel in treating the same type of fracture using currently available means for such treatment.

Further, aspects of the present invention to provide a pelvic compression positioning device for easy insertion of an orthotic device under a patient's fractured body part, such as a pelvis, without the need to lift the fractured body part, while being able to remove the device once the bracing portion extends on both sides of the fractured body part to enable fastening and securing of the bracing portion around the patient's fractured body part via a fastening device.

Additional advantages and novel features of the invention will be set forth in part in the description that follows, and in part will become more apparent to those skilled in the art upon examination of the following or upon learning by practice of the invention.

### BRIEF DESCRIPTION OF THE FIGURES

FIG. 1 shows an aspect of the pelvic compression positioning device of the present invention for use in conjunction with orthotic devices for partial or substantial immobilization of bone fractures;

FIGS. 2A and 2B show the pelvic compression positioning device of the present invention being detachably connected/disconnected to a bracing portion of the orthotic device, in accordance with aspects of the present invention;

FIG. 3 shows the pelvic compression positioning device in accordance with an aspect of the present invention; and

FIG. 4 shows the pelvic compression positioning device in accordance with another aspect of the present invention.

### DETAILED DESCRIPTION

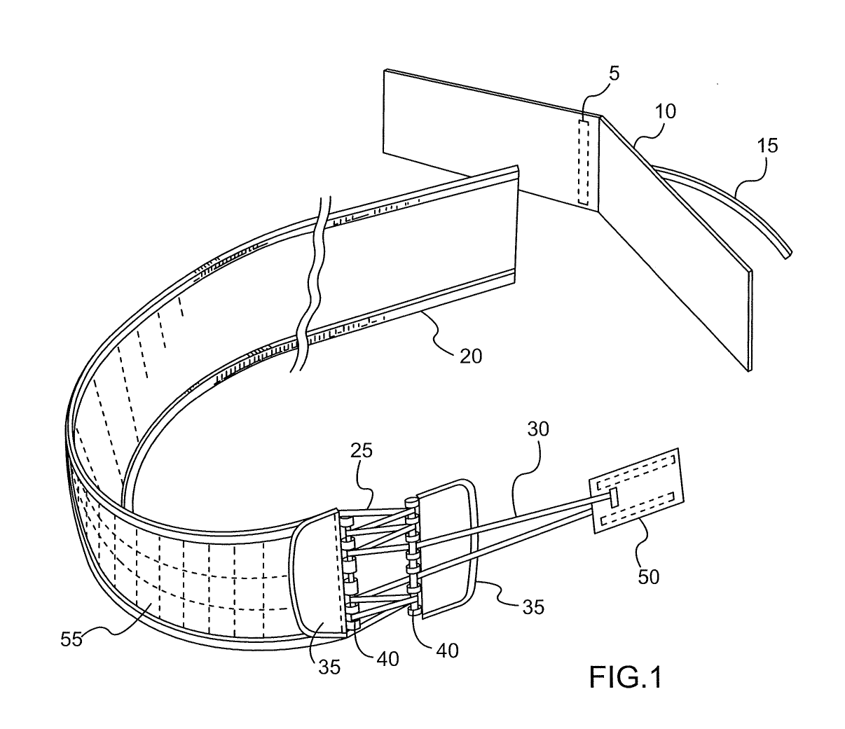

Aspects of the present invention relate to a pelvic compression positioning device for use in conjunction with orthotic devices for the emergency treatment of bone fractures. Referring now to FIG. 1, therein shown is a device comprising an elongated friction shield or envelope 10 attached to an extension handle 15, the friction shield 10 being releasably connected to one end of the bracing portion of the orthotic device 20. In operation, the extension handle 15 may be slid underneath the fractured body part of the patient and pulled out on the other side of the fractured body part, without the necessity of lifting or otherwise moving the patient. The extension handle 15 may be made of a resilient yet pliable material, such as plastic or metal, to ensure that it easily slides under the body of the patient without the need to lift the patient. In accordance with an aspect, any other resilient yet pliable material may be used, which ensures that the extension handle 15 regains its original shape, is sufficiently stiff to be inserted under a patient and pushed through to the other side, and strong enough to be pulled through guiding the friction shield 10.

In accordance with one aspect, the extension handle 15 may be curved and may have a length of 20 inches or more to ensure that it may be inserted under one side of the fractured body part, such as a pelvis, and protrude at the other side, regardless of the patient's body size. Once the extension handle 15 is inserted underneath the fractured body part of the patient, and extends at the other side, it may be pulled away from the other side, thereby ensuring that the attached friction shield or envelope 10, detachably connected to the bracing portion of the orthotic device 20, is slid underneath the patient, without the need for lifting or substantially disturbing the patient or the patient's position. Upon sliding the friction shield 10, detachably connected to the bracing portion 20, underneath the body of the patient such that it completely extends away from the opposite side of the patient, the friction shield 10 and extension handle 15 may be detached from the bracing portion 20, such that the bracing portion 20 is extended underneath the body of the patient. In accordance with one aspect, the friction shield 10 and extension handle 15 may remain attached to the bracing portion 20.

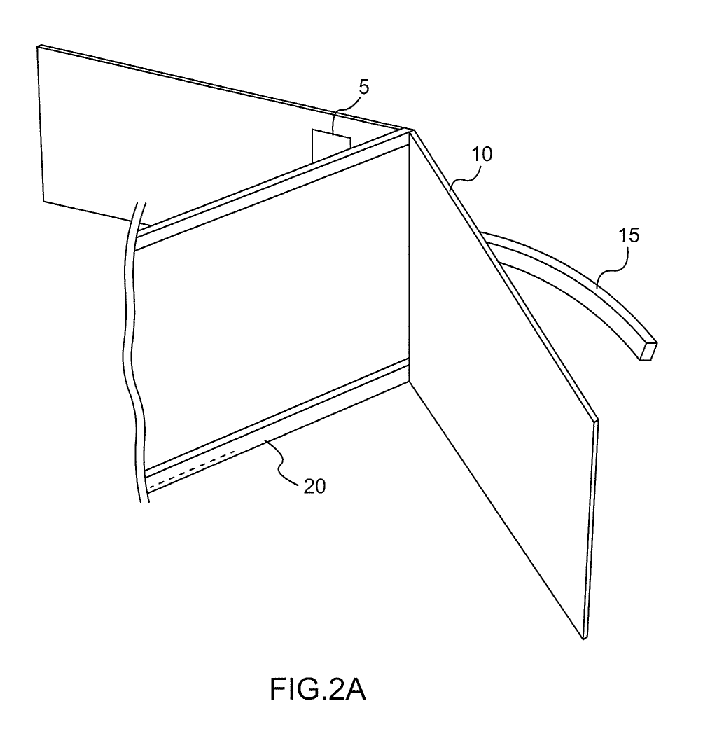

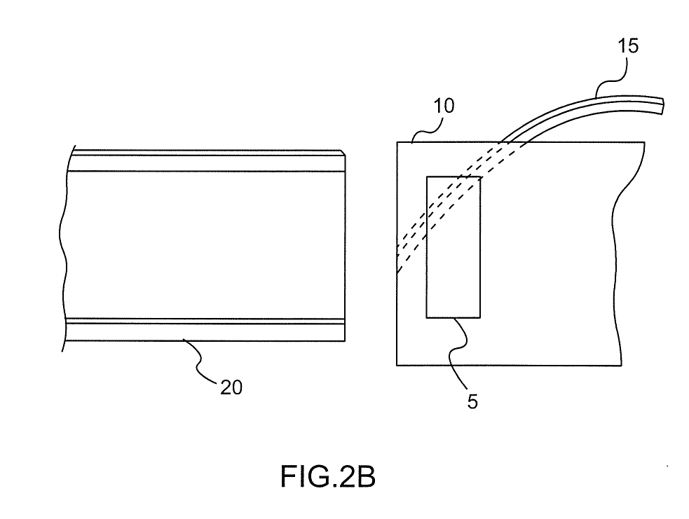

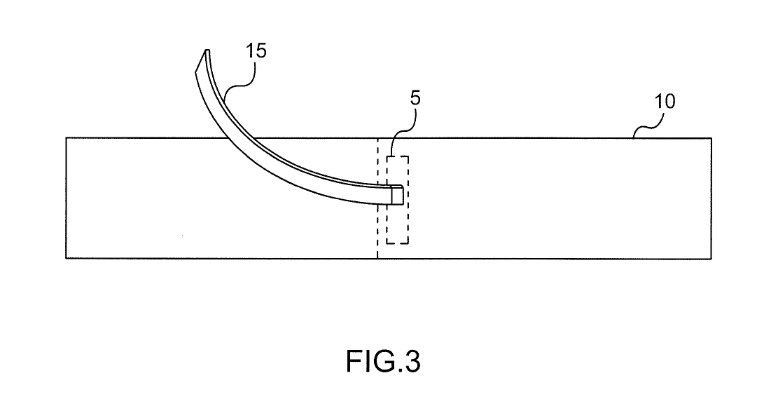

As shown in FIG. 2A, the extension handle 15 may be attached to a central portion of the friction shield 10, such that the friction shield 10 may be folded over the bracing portion of the orthotic device 20 and may be detachably connected to one or both sides of the bracing portion 20, in accordance with one aspect. In accordance with another aspect, as shown in FIG. 2B, the extension handle may be attached to a distal portion of the friction shield.

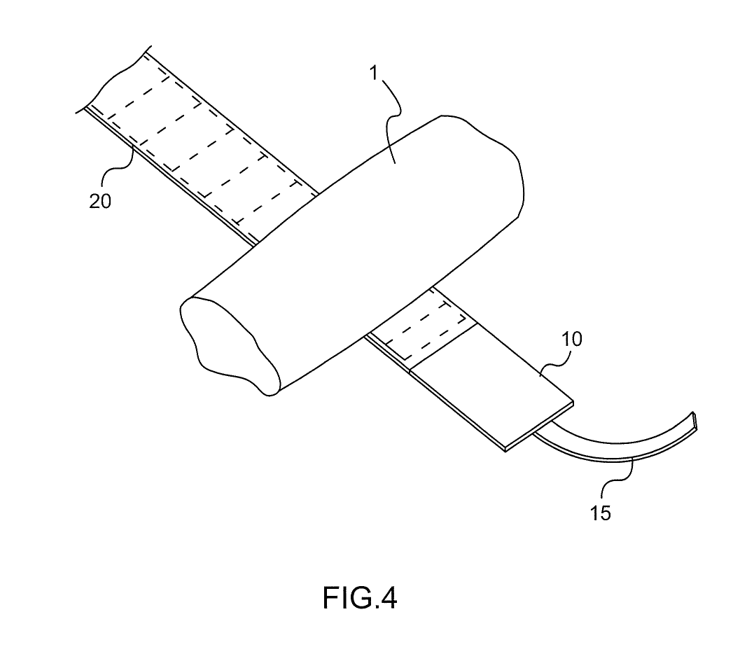

The bracing portion 20 may be cut to the proper length with a scissors, such as the type commonly used in cutting adhesive tape and gauze, or other cutting instrument. In accordance with one aspect, when the distal (interchangeably referred to herein as free) ends of the bracing portion 20 are wrapped around the patient's fractured body part (see fractured body part 1 in FIG. 4), there may be a gap between the free ends of the bracing portion 20. In accordance with another aspect, there may be no gap, or there may be an overlap of the free ends of the bracing portion 20. A detachable fastening device 35 may then be detachably secured to each of the free ends of the bracing portion 20, and suitably tightened to provide the appropriate amount of compression. The detachable spindle 40 and ribbon 25 (interchangeably referred to as tape or webbing herein) assembly shown in FIG. 1 has the added advantage of reducing patient movement and the lateral force applied to the patient upon tightening the fastening device 35. This is due to the single direction of pulling of the tape or webbing 25 when tightening the device to complete the orthotic device application. As shown in FIG. 1, the tape or webbing 25 alternately passes around the rotating free floating spindles (interchangeably referred to herein as dowels) 40, and is pulled in a single direction to tighten the spindle and tape assembly and to remove the excess tape.

In known devices the extended cord is alternately hooked around extensions or tabs and insecurely stores the excess cord, while exerting some lateral force to the patient. The present invention, in one aspect, recovers excess tape or webbing 25 directly onto the extension handle 15 which is releasably attached to the exterior, while exerting force from one direction only. The single direction of pulling and storing the excess tape or webbing allows more comfort for the patient and is easier and more secure to apply than known systems.

The friction shield or envelope 10, in accordance with aspects of the present invention, may be detachably connected to the bracing portion of the orthotic device 20 via hook and loop fabric strips 5 such as Velcro ® or other similar means, for example. Buttons and button holes, snap fasteners, complimentary hook-and-loop fastener portions or other similar fasteners 5 commonly used in garments may be also be employed for this purpose. The friction shield 10 may be detachably connected to one or both sides of the bracing portion 20 of the orthotic device.

In accordance with one aspect, the material of the friction shield or envelope 10 may be any slidable material, such as synthetic fabric, polyester, pliable plastic, or laminated cloth, which enables easy sliding of the friction shield underneath a fractured body part of a patient when pulled by the extension handle. The friction shield 10 material may be made up of one or more layers, with only the outer layer being of slidable material. However, the friction shield should be thin enough to be easily slid underneath the patient when pulled via the extension handle 15. According to another aspect, the friction shield 10 material may be slidable yet rigid, such that it may be bent when a sufficient force is applied to a surface of the material. In accordance with one aspect, the friction shield 10 material resumes its original configuration when the deforming force is removed. In accordance with one aspect, the friction shield 10 material may be laminated breathable fabric, which may be longitudinally stable but laterally flexible, such that is conforms to various body parts of a patient, while being slid underneath the patient. An example of this type of fabric is manufactured by Velcro ® USA.

The friction shield 10 may have a width that is the same as, or larger than, the width of the bracing portion 20. In accordance with one aspect, the width of the friction shield 10 may be less than the width of the bracing portion 20. In accordance with one aspect, the friction shield material longitudinally stable, so that it does not stretch.

Further, in accordance with one aspect, the friction shield or envelope 10 may be cut to a more manageable size using a scissors, for example, if the patient's fractured body part is of a smaller size.

While this invention has been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects of the invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents.

1. A pelvic compression positioning device comprising:
an extension handle;
a friction shield attached to the extension handle, the friction shield being releasably connected to a bracing portion of an orthotic device,
wherein, the extension handle and the friction shield are slid underneath a fractured body part of a patient without substantially moving the fractured body part.

2. The pelvic compression device of claim 1, further comprising:
a detachable fastening device provided at at least one free end of the bracing portion configured to detachably secure the bracing portion around the fractured body part.

3. The pelvic compression device of claim 2, wherein the detachable fastening device further comprises:
a rod and tape assembly configured to tighten the detachable fastening device.

4. The pelvic compression device of claim 3, wherein the detachable fastening device is tightened by pulling the tape in a single direction.

5. The pelvic compression device of claim 1, wherein the friction shield has one or more layers, and an outer layer of a slidable material.

6. The pelvic compression device of claim 1, wherein the extension handle has a curved configuration.

7. The pelvic compression device of claim 1, wherein the extension handle has a length of at least 20 inches.

8. The pelvic compression device of claim 1, wherein the friction shield is releasably connected to the bracing portion of an orthotic device via a hook and loop fastener.

9. A method of performing pelvic compression, the method comprising:
releasably connecting a friction shield attached to an extension handle to a bracing portion of an orthotic device having two free ends ;
inserting the extension handle beneath a fractured body part from one direction;
pulling the extension handle beneath the fractured body part from another direction, such that the releasably connected bracing portion is slid underneath the fractured body part;
detaching the friction shield from the bracing portion; and
connecting the two free ends of the bracing portion of the orthotic device.

10. The method of claim 9, wherein connecting the two free ends is performed via a detachable fastening device.

11. The method of claim 10, wherein the detachable fastening device comprises a rod and tape assembly configured to tighten the detachable fastening device.

12. The method of claim 11, wherein the detachable fastening device is tightened by pulling the tape in a single direction.

13. The method of claim 9, further comprising:
cutting one or both free ends of the bracing portion when the two free ends overlap.

14. The method of claim 9, wherein the friction shield has one or more layers, and an outer layer of a slidable material.

15. The method of claim 9, wherein the friction shield is releasably connected to the bracing portion of an orthotic device via a hook and loop fastener.

16. The method of claim 9, wherein the extension handle has a curved configuration.

17. The method of claim 9, wherein the extension handle has a length of at least 20 inches.
